(19) United States
(12) Patent Application Publication
(10) Pub. No.: US 2016/0151489 A1
(43) Pub. Date: Jun. 2, 2016

(54) COMBINATION OF A LIGAND OF HVEM AND AN IMMUNOTOXIN FOR USE IN THERAPY

(71) Applicants: INSERM (INSTITUT NATIONAL DE LA SANTÉ ET DE LA RECHERCHE MÉDICALE), Paris (FR); ASSISTANCE PUBLIQUE HOPITAUX DE MARSEILLE, MARSEILLE cedex (FR); INSTITUT JEAN PAOLI & IRENE CALMETTES, Marseille (FR); UNIVERSITÉ D'AIX-MARSEILLE, Marseille Cedex 07 (FR); CENTRE NATIONAL DE LA RECHERCHE SCIENTIFIQUE (CNRS), Paris (FR)

(72) Inventors: Daniel OLIVE, Marseille (FR); Christine PASERO, Marseille (FR)

(21) Appl. No.: 14/905,842

(22) PCT Filed: Jul. 18, 2014
(86) PCT No.: PCT/EP2014/065512

§ 371 Date: Jan. 18, 2016

(30) Foreign Application Priority Data
Jul. 19, 2013 (EP) 13306037.6

(51) Int. Cl. A61K 39/395; A61K 38/16; C07K 16/30

(52) U.S. Cl. CPC A61K 39/39558; C07K 16/3069; A61K 38/168; C07K 2317/77; A61K 2039/505

(57) ABSTRACT

The invention relates to i) a ligand of HVEM, and ii) an immunotoxin, as a combined preparation for simultaneous, separate or sequential use in the treatment of a solid tumor.

### FIELD OF THE INVENTION

The present invention relates to i) a ligand of HVEM, and ii) an immunotoxin, as a combined preparation for simultaneous, separate or sequential use in the treatment of a solid tumor. The invention also relates to a pharmaceutical composition comprising i) a ligand of HVEM and an immunotoxin ii).

### BACKGROUND OF THE INVENTION

Treatment of solid tumors is a major concern of public health. Over the past 30 years, fundamental advances in the chemotherapy of neoplastic diseases have been realized. However, despite the impressive advances that have been made, many of the most prevalent forms of human cancers, for example, solid tumors of the brain, prostate or breast, are still difficult to treat efficiently.

Brain tumors are very often fatal. Chemotherapy is often ineffective against these tumors due, in large part, to the difficulty in achieving therapeutically effective levels of chemotherapeutic agents in the area of tumor growth and infiltration. For example, the existence of the blood brain barrier can restrict the flow of certain chemotherapeutic agents from the cerebral capillaries to the brain. Methods of treating brain tumors have included the delivery of chemotherapeutics directly to the surgical cavity resulting from surgical debulking of the tumor or intratumorally.

Even solid tumor therapies, which are not restricted by the existence of the blood brain barrier, can give unsatisfactory results. For example, prostate cancer is a common form of cancer among males, and there are cases of aggressive prostate cancers. Clinical evidence shows that human prostate cancer has the propensity to metastasize to bone and lymph nodes and is currently in the USA the second leading cause of cancer death, after lung cancer, among men. Commonly, treatment is based on surgery and/or radiation therapy and/or chemotherapy, but these methods give unsatisfactory results in a significant percentage of cases.

Pancreatic cancer is another example of solid tumor. It has one of the highest mortality rates of any malignancy, and it is the fourth most common cause of cancer-related deaths in the USA. The poor prognosis of this malignancy is a result of the difficulty of early diagnosis and poor response to current therapeutic methods.

Thus, it is clear that there is a need for improvements in current therapies for the treatment of cancers involving solid tumors.

### SUMMARY OF THE INVENTION

The inventors have shown for the first time that HVEM is expressed on solid tumor cells, and that the combination of a ligand of HVEM, particularly an anti-HVEM antibody, and an immunotoxin is efficient for inducing tumor cell death. Therefore, the inventors have developed a new highly promising strategy for use in therapy.

A first object of the invention thus relates to i) a ligand of HVEM, and ii) an immunotoxin, as a combined preparation for simultaneous, separate or sequential use in the treatment of a solid tumor.

The invention also relates to i) a ligand of HVEM, and ii) an immunotoxin, for use in the treatment of a solid tumor.

The invention also relates to a pharmaceutical composition comprising i) a ligand of HVEM, and ii) an immunotoxin.

The ligand of HVEM is preferably chosen in the group consisting of LIGHT, LTa, BTLA, CD160, HSV-gD, and anti-HVEM antibodies, fragments thereof and derivatives thereof.

Said anti-HVEM antibody is preferably a monoclonal antibody chosen from the monoclonal antibody obtainable from the hybridoma deposited at the Collection Nationale de Cultures de Microorganismes on Apr. 26, 2007, under the number CNCM I-3752, the monoclonal antibody obtainable from the hybridoma deposited at the Collection Nationale de Cultures de Microorganismes on Apr. 26, 2007, under the number CNCM I-3753, the monoclonal antibody obtainable from the hybridoma deposited at the Collection Nationale de Cultures de Microorganismes on Apr. 26, 2007, under the number CNCM I-3754 and the monoclonal antibody obtainable from the hybridoma deposited at the Collection Nationale de Cultures de Microorganismes on May 16, 2013, under the number CNCM I-4751.

The invention also relates to the hybridoma cell line deposited at the Collection Nationale de Cultures de Microorganismes on May 16, 2013, under the number CNCM I-4751. The invention finally relates to a monoclonal antibody obtainable from the hybridoma deposited at the Collection Nationale de Cultures de Microorganismes on May 16, 2013, under the number CNCM I-4751.

### DETAILED DESCRIPTION OF THE INVENTION

A first object of the invention thus relates to i) a ligand of HVEM, and ii) an immunotoxin, as a combined preparation for simultaneous, separate or sequential use in the treatment of a solid tumor.

The invention also relates to a pharmaceutical composition comprising i) a ligand of HVEM, and ii) an immunotoxin. Said pharmaceutical composition may be used in therapy, particularly in the treatment of a solid tumor.

The ligand of HVEM is preferably chosen in the group consisting of LIGHT, LTa, BTLA, CD160, HSV-gD, and anti-HVEM antibodies, fragments thereof and derivatives thereof.

Preferably, said ligand of HVEM is chosen from anti-HVEM antibodies, fragments thereof and derivatives thereof.

The inventors have demonstrated in the examples that HVEM is expressed on solid tumor cells, and may thus be a marker of these cells. When the combination of an anti-HVEM antibody and an immunotoxin is used, especially an immunotoxin made up of an antibody portion linked to saporin, tumor cell death is induced. Thus, the combination of both actives may be useful for the treatment of solid tumors. Said scheme of action is presented on FIG. 3.

The term “HVEM”, as used herein, is intended to encompass all synonyms including, but not limited to, “Herpes Virus Entry Mediator”, “HVEA”, “Herpes Virus Entry Mediator A”, “TNFRSF14”, “Tumor Necrosis Factor Receptor Superfamily Member 14”, “TNR14”, “LIGHTR”, “LIGHT receptor”, “TR2”, “TNF Receptor-like”, “ATAR”, “Another TRAF-Associated Receptor”. TNFRSF14 is the HUGO (Human Genome Organization) Gene Nomenclature Committee (HGNC) approved symbol. The UniProtKB/Swiss-Prot “Primary Accession Number” for HVEM is Q92956. The “Secondary Accession Numbers” are Q8WXR1, Q96J31 and Q9UM65.

By “ligand” is meant a natural or synthetic compound which binds to HVEM to form a HVEM-ligand complex.

So far, four ligands have been identified which bind to HVEM. Two of these ligands, LIGHT and LTα (lymphotoxin-alpha), are member of the TNF family of molecules (Morel, Y. et al., 2000; Mauri, D. N. et al., 1998 and Harrop, J. A. et al., 1998). Structurally, members of the TNF family are generally expressed as single-pass type 2 transmembrane, homotrimer or heterotrimer, glycoproteins. Following their expression as transmembrane proteins, they are cleaved by proteolytic action to produce a soluble form of the ligand. The third ligand for HVEM, BTLA, a type 1 transmembrane glycoprotein, is a member of the immunoglobulin (Ig) superfamily of molecules and is closely related to CD28 (Gonzalez, L. C. et al., 2005). The fourth ligand, glycoprotein D (gD), is a structural component of the herpes simplex virus (HSV) envelope, and is essential for HSV entry into host cells (Montgomery, R. I. et al., 1996; Hsu, H. et al., 1997; Kwon, B. S. et al., 1997; Tan, K. B. et al., 1997; Marsters, S. A. et al., 1997; Wallach, D. et al., 1999; Collette, Y. et al., 2003; Harrop, J. A. et al., 1998; Gonzalez, L. C. et al., 2005 and Whitbeck, J. C. et al., 1997).

Binding studies (Gonzalez, L. C. et al., 2005 and Sedy, J. R. et al., 2005) which were later supported by crystallography (Compaan, D. M. et al., 2005) indicate that BTLA interacts with the most membrane-distal CRD region of HVEM. The membrane-distal CRD1 region of HVEM has also been implicated in the interactions with HSV-gD, with additional contributions from CRD2 (Compaan, D. M. et al., 2005 and Carfi, A. et al., 2001). Despite the sequence and structural dissimilarities between BTLA and HSV-gD, the crystal structure studies also show that their binding sites on HVEM cover largely overlapping surfaces (Compaan, D. M. et al., 2005 and Carfi, A. et al., 2001)

The term “LIGHT”, as used herein, is intended to encompass all synonyms including, but not limited to, “lymphotoxin-like, exhibits inducible expression, competes with herpes simplex virus glycoprotein D for HVEM, a receptor expressed by T lymphocytes”, “TNFSF14”, “Tumor Necrosis Factor Ligand Superfamily Member 14”, “TNF14_HUMAN”, “HVEM-L”, “HVEML”, “HVEM-Ligand”, “Herpes Virus Entry Mediator Ligand”, “Herpesvirus entry mediator-ligand”, “TL4”, “TNF-like 4”, “TN14”, “LTγ” and “CD258”. TNFSF14 is the HGNC approved symbol. CD258 is the cluster designation assignment of the HLDA (Human Leukocyte Differentiation Antigens) Workshop. The UniProtKB/Swiss-Prot “Primary Accession Number” for LIGHT is 043557. The “Secondary Accession Numbers” are 075476, Q8WVF8 and Q96LD2.

As used herein, the term “solid tumor” refers to an abnormal mass or population of cells that result from excessive cell division, whether malignant or benign, and all pre-cancerous and cancerous cells and tissues.

Examples of solid tumors include prostate cancer, pancreatic cancer, breast cancer, melanoma, B cell lymphoma, brain cancer, bladder cancer, colon cancer, intestinal cancer, lung cancer, stomach cancer, cervical cancer, ovarian cancer, liver cancer, skin cancer, colorectal cancer, endometrial carcinoma, salivary gland carcinoma, kidney cancer, thyroid cancer, various types of head and neck cancers.

Preferably, said solid tumor is selected among prostate and pancreatic cancers.

The ligand of HVEM i) may be an anti-HVEM antibody, a fragment thereof or a derivative thereof. Typically, said anti-HVEM antibody is chosen among polyclonal antibody, monoclonal antibody, chimeric antibody, humanized antibody, antibody fragments and antibody derivatives.

Preferably, said anti-HVEM antibody is a monoclonal antibody.

As used herein, the term “human antibody” refers to an antibody in which a substantial portion of the antibody molecule resembles, in amino acid sequence or structure, that of an antibody derived from human origin. The term “humanized antibody” refers to an antibody which has been modified by genetic engineering or by other means to be similar in structure or amino acid sequence to naturally occurring human antibodies. A “human antibody” or a “humanized antibody” may be considered more suitable in instances where it is desirable to reduce the immunogenicity of the antibody for administration to humans for therapeutic, prophylactic or diagnostic purposes.

Antibodies specifically directed against HVEM may be derived from a number of species including, but not limited to, rodent (mouse, rat, rabbit, guinea pig, hamster, and the like), porcine, bovine, equine or primate and the like. Antibodies from primate (monkey, baboon, chimpanzee, etc.) origin have the highest degree of similarity to human sequences and are therefore expected to be less immunogenic. Antibodies derived from various species can be “humanized” by modifying the amino acid sequences of the antibodies while retaining their ability to bind the desired antigen. Antibodies may also be derived from transgenic animals, including mice, which have been genetically modified with the human immunoglobulin locus to express human antibodies. Procedures for raising “polyclonal antibodies” are well known in the art. For example, polyclonal antibodies can be obtained from serum of an animal immunized against HVEM, which may be produced by genetic engineering for example according to standard methods well-known by one skilled in the art. Typically, such antibodies can be raised by administering HVEM protein subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times at six weeks' interval. A sample of serum is then collected 10 days after each boost. Polyclonal antibodies are then recovered from the serum by affinity chromatography using the corresponding antigen to capture the antibody. This and other procedures for raising polyclonal antibodies are disclosed by (Harlow et al., 1988), which is hereby incorporated in the references.

Although historically monoclonal antibodies were produced by immortalization of a clonally pure immunoglobulin secreting cell line, a monoclonally pure population of antibody molecules can also be prepared by the methods of the present invention. Laboratory methods for preparing monoclonal antibodies are well known in the art (see, for example, Harlow et al., 1988).

A “monoclonal antibody” or “mAb” in its various names refers to a population of antibody molecules that contains only one species of antibody combining site capable of immunoreacting with a particular epitope. A monoclonal antibody thus typically displays a single binding affinity for any epitope with which it immunoreacts. Monoclonal antibody may also define an antibody molecule which has a plurality of antibody combining sites, each immunospecific for a different epitope. For example, a bispecific antibody would have two antigen binding sites, each recognizing a different interacting molecule, or a different epitope. As used herein, the terms “antibody fragment”, “antibody portion”, “antibody variant” and the like include any protein or polypeptide containing molecule that comprises at least a portion of an immunoglobulin molecule such as to permit specific interaction between said molecule and an antigen (e.g. HVEM). The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of a ligand or counter-receptor (e.g. LIGHT, BTLA or HSV-gD) which can be incorporated into an antibody of the present invention to permit interaction with the antigen (e.g. HVEM).

Monoclonal antibodies (mAbs) may be prepared by immunizing a mammal such as mouse, rat, primate and the like, with purified HVEM protein. The antibody-producing cells from the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in (Kohler and Milstein, 1975). Alternatively, the immunoglobulin genes may be isolated and used to prepare a library for screening for reactive specifically reactive antibodies. Many such techniques including recombinant phage and other expression libraries are known to one skilled in the art.

While mAbs can be produced by hybridoma culture the invention is not to be so limited. Also contemplated is the use of mAbs produced by cloning and transferring the nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

In a particular embodiment, mAbs recognizing HVEM may be generated by immunization of Balb-c mice with the respective recombinant human Fc-IgG1 fusion proteins. Spleen cells were fused with X-63 myeloma cells and cloned according to already described procedures (Olive D, 1986). Hybridoma supernatants were then screened by staining of transfected cells and for lack of reactivity with untransfected cells.

Antibody generation techniques not involving immunisation are also contemplated such as for example using phage display technology to examine naive libraries (from non-immunised animals); see (Barbas et al., 1992, and Waterhouse et al. (1993). Antibodies of the invention are suitably separated from the culture medium by conventional immunoglobulin purification procedures such as, for example, affinity, ion exchange and/or size exclusion chromatography, and the like.

In a particular embodiment, the antibody of the invention may be a human chimeric antibody. Said human chimeric antibody of the present invention can be produced by obtaining nucleic sequences encoding VL and VH domains, constructing a human chimeric antibody expression vector by inserting them into an expression vector for animal cell having genes encoding human antibody CH and human antibody CL, and expressing the expression vector by introducing it into an animal cell. The CH domain of a human chimeric antibody may be any region which belongs to human immunoglobulin, but those of IgG class are suitable and any one of subclasses belonging to IgG class, such as IgG1, IgG2, IgG3 and IgG4, can also be used. Also, the CL of a human chimeric antibody may be any region which belongs to Ig, and those of kappa class or lambda class can be used. Methods for producing chimeric antibodies involve conventional recombinant DNA and gene transfection techniques are well known in the art (See Morrison S L. et al. (1984) and patent documents U.S. Pat. No. 5,202,238; and U.S. Pat. No. 5,204,244).

In another particular embodiment, said antibody may be a humanized antibody. Said humanized antibody may be produced by obtaining nucleic acid sequences encoding for CDRs domain by inserting them into an expression vector for animal cell having genes encoding a heavy chain constant region identical to that of a human antibody; and a light chain constant region identical to that of a human antibody, and expressing the expression vector by introducing it into an animal cell. The humanized antibody expression vector may be either of a type in which a gene encoding an antibody heavy chain and a gene encoding an antibody light chain exist on separate vectors or of a type in which both genes exist on the same vector (tandem type). In respect of easiness of construction of a humanized antibody expression vector, easiness of introduction into animal cells, and balance between the expression levels of antibody H and L chains in animal cells, a tandem type of the humanized antibody expression vector is more preferable (Shitara K et al. 1994). Examples of the tandem type humanized antibody expression vector include pKANTEX93 (WO 97/10354), pEE18 and the like. Methods for producing humanized antibodies based on conventional recombinant DNA and gene transfection techniques are well known in the art (See, e.g. Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A (1991); Studnicka G M et al. (1994); Roguska M A. et al. (1994)), and chain shuffling (U.S. Pat. No. 5,565,332). The general recombinant DNA technology for preparation of such antibodies is also known (see European Patent Application EP 125023 and International Patent Application WO 96/02576).

Preferably, the anti-HVEM antibodies are chosen from:
antibodies which bind to HVEM and do not inhibit the binding of BTLA to HVEM, and
antibodies which inhibit the binding of BTLA to HVEM and which bind to HVEM on an epitope different from the human HVEM sequence CPKCSPGYRVKEACGELTGTVCEPC (SEQ ID NO:1).

In a preferred embodiment said anti-HVEM antibody or said fragment is a monoclonal antibody (mAb) or a fragment thereof which recognizes an epitope selected from the group consisting of groups I, II, III, IV, V or VI defined below.

The 6 distinct groups of mAbs are the following:
1. Group I: mAbs which do not bind to the CRD1 deletion mutant but are affected by the de1129-133 deletion mutant, and only block the binding of HVEM to LIGHT.
2. Group II: mAbs which bind to the CRD1 deletion mutant, but not to the de1129-133 deletion mutant or to the mut131-133 mutant.
3. Group III: mAbs which do not bind to the CRD1 deletion mutant and are not affected by the de1129-133 deletion mutant, and do not inhibit the binding of the three HVEM ligands (LIGHT, BTLA and CD160).
4. Group IV: mAbs which are not affected by the CRD1 deletion mutant but are affected by the de1129-133 deletion mutant, and do not inhibit the binding of the three HVEM ligands (LIGHT, BTLA and CD160).
5. Group V: mAbs which bind to the CRD1 deletion mutant but not to the de1129-133 deletion mutant, which are not affected by the mut131-133 mutant, and which are not able to block HVEM binding to the three ligands (LIGHT, BTLA and CD160).
6. Group VI: mAbs which bind to the CRD1 deletion mutant but are affected by the de1129-133 deletion mutant, or by the mut131-133 mutant, and are able to block HVEM binding to all ligands (LIGHT, BTLA and CD160).

The three HVEM mutants described above correspond to the HVEM sequence with the following modifications:

i) Two deletion mutants:
a. the CRD1 deletion mutant corresponds to the deletion of the CRD1 domain, and
b. the de1129-133 deletion mutant corresponds to the deletion of amino acids 129-133 within the CRD3 domain;
ii) A substitution mutant: the mut131-133 mutant corresponds to the substitution of residues 131-133 by three alanine residues.

Preferably, the anti-HVEM antibody fragments are chosen from Fab (e.g., by papain digestion), Fab′ (e.g., by pepsin digestion and partial reduction), F(ab)2, F(ab′)2 (e.g., by pepsin digestion) and dAb fragments.

Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Said Fab fragment of the present invention can be obtained by treating an antibody which specifically reacts with human HVEM with a protease, papaine. Also, the Fab may be produced by inserting DNA encoding Fab of the antibody into a vector for prokaryotic expression system or for eukaryotic expression system, and introducing the vector into a procaryote or eucaryote to express the Fab.

Said F(ab′)₂ of the present invention may be obtained by treating an antibody which specifically reacts with HVEM with a protease, pepsin. Also, the F(ab′)₂ can be produced by binding Fab′ described below via a thioether bond or a disulfide bond. Said Fab′ may be obtained by treating F(ab′)₂ which specifically reacts with HVEM with a reducing agent, dithiothreitol. Also, the Fab′ can be produced by inserting DNA encoding Fab′ fragment of the antibody into an expression vector for prokaryote or an expression vector for eukaryote, and introducing the vector into a prokaryote or eukaryote to effect its expression.

Preferably, the anti-HVEM antibody derivatives are chosen from scFv, (scFv)2, diabodies, multimeric scFv derived from an anti-HVEM antibody and fused to a Fc fragment, whole anti-HVEM antibodies linked together to reach an aggregated form, and antibodies containing at least two Fabs bound face-to-tail.

Said scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. No. 5,859,205; U.S. Pat. No. 5,585,089; U.S. Pat. No. 4,816,567; EP0173494).

In a particular embodiment, monoclonal antibodies of the invention are monovalent, bivalent, multivalent, monospecific, bispecific, or multispecific. In another preferred embodiment, the antibody directed against HVEM is a binding fragment or a conjugate. For examples antibodies of the invention may be conjugated to a growth inhibitory agent, cytotoxic agent, or a prodrug-activating enzyme.

It may be also desirable to modify the antibody of the invention with respect to effector functions, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron P C. et al. 1992; and Shopes B. 1992) Another type of amino acid modification of the antibody of the invention may be useful for altering the original glycosylation pattern of the antibody.

By “altering” is meant deleting one or more carbohydrate moieties found in the antibody, and/or adding one or more glycosylation sites that are not present in the antibody.

Glycosylation of antibodies is typically N-linked. “N-linked” refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites).

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. For example, such methods are described in WO87/05330.

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody comprises linking the antibody to one of a variety of non-proteinaceous polymers, e.g. polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

In a preferred embodiment said anti-HVEM antibody is a monoclonal antibody obtainable from the hybridoma deposited at the Collection Nationale de Cultures de Microorganismes (CNCM, Institut Pasteur, 25 rue du Docteur Roux, 75724 Paris Cedex 15, France), in accordance with the terms of Budapest Treaty, on Apr. 26, 2007, under the number CNCM I-3752.

As used herein, the expression “HVEM 4.4” refers to an isolated HVEM antibody which is obtainable from the hybridoma accessible under CNCM deposit number I-3752.

In a preferred embodiment said anti-HVEM antibody is a monoclonal antibody obtainable from the hybridoma deposited at the Collection Nationale de Cultures de Microorganismes (CNCM, Institut Pasteur, 25 rue du Docteur Roux, 75724 Paris Cedex 15, France), in accordance with the terms of Budapest Treaty, on Apr. 26, 2007, under the number CNCM I-3753.

As used herein, the expression “HVEM 11.8” refers to an isolated HVEM antibody which is obtainable from the hybridoma accessible under CNCM deposit number I-3753.

In a preferred embodiment said anti-HVEM antibody is a monoclonal antibody obtainable from the hybridoma deposited at the Collection Nationale de Cultures de Microorganismes (CNCM, Institut Pasteur, 25 rue du Docteur Roux, 75724 Paris Cedex 15, France), in accordance with the terms of Budapest Treaty, on Apr. 26, 2007, under the number CNCM I-3754.

As used herein, the expression “HVEM 20.4” refers to an isolated HVEM antibody which is obtainable from the hybridoma accessible under CNCM deposit number I-3754.

In a preferred embodiment said anti-HVEM antibody is a monoclonal antibody obtainable from the hybridoma deposited at the Collection Nationale de Cultures de

Microorganismes (CNCM, Institut Pasteur, 25 rue du Docteur Roux, 75724 Paris Cedex 15, France), in accordance with the terms of Budapest Treaty, on May 16, 2013, under the number CNCM I-4751.

As used herein, the expression “HVEM 14.9” refers to an isolated HVEM antibody which is obtainable from the hybridoma accessible under CNCM deposit number I-4751.

The term “hybridoma” denotes a cell, which is obtained by subjecting a B cell, prepared by immunizing a non-human mammal with an antigen, to cell fusion with a myeloma cell derived from a mouse or the like which produces a desired monoclonal antibody having an antigen specificity.

According to the invention, said ligand of HVEM, is associated with an immunotoxin ii).

By “immunotoxin”, it is meant a chimeric protein made of a modified antibody or antibody fragment (also called in the present application “secondary antibody”), attached to a fragment of a toxin. The modified antibody or antibody fragment of the immunotoxin is covalently attached to the fragment of a toxin. Preferably, the fragment of the toxin is linked by a linker to the antibody or fragment thereof. Said linker is preferably chosen from 4-mercaptovaleric acid and 6-maleimidocaproic acid.

Typically, said modified antibody or antibody fragment comprises a Fv portion, and targets the ligand of HVEM, preferably the anti-HVEM antibody, its fragment or derivative. Said modified antibody or antibody fragment is thus able to bind to the ligand of HVEM, on an epitope different from that of the ligand of HVEM.

Said immunotoxin ii) also comprises a toxin or a fragment thereof. Preferably, said toxin or its fragment is a Ribosome Inactivating Protein (RIP).

Preferably, the Ribosome Inactivating Protein is chosen from saporin, ricin, abrin, gelonin, Pseudomonas exotoxin (or exotoxin A), trichosanthin, luffin, agglutinin and the diphtheria toxin. More preferably, the toxin is saporin.

Preferably, the toxin may also be a chemical drug.

Preferably, the toxin is chosen from modeccin, mitogellin, chlortetracycline, mertansine, monomethyl auristatin E, monomethyl auristatin F, and enediynes, especially calicheamicins (like calicheamicin k or calicheamicin γ1) and their related esperamicins (like esperamicin A1). Enediynes are chemical compounds characterized by either 9- or 10-membered rings containing two triple bonds separated by a double bond.

When the toxin is mertansine, it is linked to the antibody or a fragment thereof by a linker. When the linker is 4-mercaptovaleric acid, the group comprising the toxin and the linker is called emtansine.

When the toxin is monomethyl auristatin E (MMAE), it is linked to the antibody or a fragment thereof by a structure comprising a spacer (which is preferably paraaminobenzoic acid), a cathepsin-cleavable linker (preferably consisting of citrulline and valine) and an attachment group or linker (preferably consisting of 6-maleimidocaproic acid). Preferably in such a case, the group comprising the toxin and the structure as defined in the previous sentence is vedotin.

When the toxin is monomethyl auristatin F (MMAF), it is linked to the antibody or a fragment thereof by a structure comprising an attachment group or linker (preferably consisting of 6-maleimidocaproic acid). Preferably in such a case, the group comprising the toxin and the structure as defined in the previous sentence is mafodotin.

The toxin may also be chosen from anticancer agents. Said anticancer agents are preferably chosen from combrestatin, colchicine, actinomycine, duocarmycins and their synthetic analogues (adozelesin, bizelesin and carzelesin), fludarabine, gemcitabine, capecitabine, methotrexate, taxol, taxotere, mercaptopurine, thioguanine, hydroxyurea, cytarabine, cyclophosphamide, ifosfamide, platinum complexes (such as cisplatin, carboplatin and oxaliplatin), mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epimbicin, 5-fluorouracil, taxanes (such as docetaxel and paclitaxel), leucovorin, levamisole, irinotecan, estramustine, etoposide, nitrogen mustards, BCNU, nitrosoureas (such as carmustine and lomustine), vinca alkaloids (such as vinblastine, vincristine, dolastatins and vinorelbine), imatinib mesylate, hexamethylenediamine, topotecan, kinase inhibitors (like the tyrosine kinase inhibitors called tyrphostins), phosphatase inhibitors, ATPase inhibitors, protease inhibitors, inhibitors of herbimycin A, genistein, erbstatin, and lavendustin A.

The toxin may also be a radioisotope, preferably chosen from ²¹¹At, ¹³¹I, ¹²⁵I, ¹⁸⁶Re, ¹⁸⁸Re, ¹⁵³Sm, P³², ⁹⁰Y, ¹⁷⁷Lu, ⁶⁷Cu, ⁴⁷Sc, ²¹²Bi, ²¹³Bi, ²²⁶Th, ¹¹¹In and ⁶⁷Ga.

Preferably, the immunotoxin is an anti-mouse IgG linked to saporin.

A further object of the invention relates to a method of treating solid tumors comprising administering in a subject in need thereof a therapeutically effective amount of a compound i) and an immunotoxin ii) as defined above.

In the context of the invention, the term “treating” or “treatment”, as used herein, means reversing, alleviating, inhibiting the progress of, or preventing the disorder or condition to which such term applies, or one or more symptoms of such a disorder or condition.

As used herein, the term “subject” denotes a mammal, such as a rodent, a feline, a canine, and a primate. Preferably a subject according to the invention is a human. According to the invention, the term “patient” or “patient in need thereof” is intended for a human or non-human mammal affected or likely to be affected by a solid tumor.

By a “therapeutically effective amount” of the ligand of HVEM i) and of the immunotoxin according to the invention is meant a sufficient amount of said antibody or said immunotoxin to treat said solid tumor, at a reasonable benefit/risk ratio applicable to any medical treatment. It will be understood, however, that the total daily usage of ligand of HVEM i) and immunotoxin ii) and compositions of the present invention will be decided by the attending physician within the scope of sound medical judgment. The specific therapeutically effective dose level for any particular patient will depend upon a variety of factors including the disorder being treated and the severity of the disorder, activity of the specific antagonist of the active employed; the specific composition employed, the age, body weight, general health, sex and diet of the patient, the time of administration, route of administration, and rate of excretion of the specific antibody employed, the duration of the treatment; drugs used in combination or coincidental with the specific polypeptide employed, and like factors well known in the medical arts. For example, it is well known within the skill of the art to start doses of the compound at levels lower than those required to achieve the desired therapeutic effect and to gradually increase the dosage until the desired effect is achieved.

A further object of the invention relates to a pharmaceutical composition comprising i) a ligand of HVEM, preferably chosen from LIGHT, LTα, BTLA, CD160, HSV-gD and anti-HVEM antibodies, fragments thereof and derivatives thereof, and ii) an immunotoxin.

Any therapeutic agent of the invention as above described may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of antagonist of the actives i) and ii) may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution may be suitably buffered and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous or intramuscular injection, other pharmaceutically acceptable forms include, e.g. tablets or other solids for oral administration; time release capsules; and any other form currently used.

Compositions of the present invention may comprise a further therapeutic active agent. The present invention also relates to a kit comprising a ligand of HVEM i) and an immunotoxin ii) as defined above and a further therapeutic active agent.

In one embodiment said therapeutic active agent is an anticancer agent. For example, said anticancer agents include but are not limited to fludarabine, gemcitabine, capecitabine, methotrexate, taxol, taxotere, mercaptopurine, thioguanine, hydroxyurea, cytarabine, cyclophosphamide, ifosfamide, platinum complexes such as cisplatin, carboplatin and oxaliplatin, mitomycin, dacarbazine, procarbizine, etoposide, teniposide, campathecins, bleomycin, idarubicin, daunorubicin, dactinomycin, plicamycin, mitoxantrone, L-asparaginase, doxorubicin, epimbicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, imatinib mesylate, hexamethylenediamine, topotecan, kinase inhibitors, phosphatase inhibitors, ATPase inhibitors, tyrphostins, protease inhibitors, inhibitors of herbimycin A, genistein, erbstatin, and lavendustin A. In one embodiment, additional anticancer agents may be selected from, but are not limited to, one or a combination of the following class of agents: alkylating agents, plant alkaloids, DNA topoisomerase inhibitors, anti-folates, pyrimidine analogs, purine analogs, DNA antimetabolites, taxanes, podophyllotoxin, hormonal therapies, retinoids, photosensitizers or photodynamic therapies, angiogenesis inhibitors, antimitotic agents, isoprenylation inhibitors, cell cycle inhibitors, actinomycins, bleomycins, anthracyclines, MDR inhibitors and Ca2+ ATPase inhibitors.

Additional anticancer agents may be selected from, but are not limited to, cytokines, chemokines, growth factors, growth inhibitory factors, hormones, soluble receptors, decoy receptors, monoclonal or polyclonal antibodies, mono-specific, bi-specific or muti-specific antibodies, monobodies, polybodies.

In the present methods for treating cancer the further therapeutic active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoemanolamine, alizapride, azasetron, benzoquinamide, bietanautine, bromopride, buclizine, clebopride, cyclizine, dimenhydrinate, diphenidol, dolasetron, meclizine, methallatal, metopimazine, nabilone, oxypemdyl, pipamazine, scopolamine, sulpiride, tetrahydrocannabinols, thiethylperazine, thioproperazine and tropisetron. In a preferred embodiment, the antiemetic agent is granisetron or ondansetron.

In still another embodiment, the other therapeutic active agent can be an opioid or non-opioid analgesic agent Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazone, pemazocine, cyclazocine, methadone, isomethadone and propoxyphene. Suitable non-opioid analgesic agents include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

The invention will be further illustrated through the following examples, figures and tables.

### FIGURES

FIG. 1: HVEM expression on prostate tumor cell lines (A) and primary prostate tumors (B).

A. Prostate tumor cell lines:

Flow cytometry expression of HVEM and its ligands BTLA, CD160 and LIGHT on PC3 and DU145 prostate tumor cell lines.

B. Human primary prostate tumors:

Human prostate tumor tissue was obtained at the day of surgery and mechanically dilacerated. Cell suspension was phenotyped by flow cytometry for the expression of HVEM on CD3-large tumor cells.

FIG. 2: Targeting through internalization of HVEM mAbs/saporin toxin complex.

A. The anti-HVEM 8.5 mAb induce caspase 3/7 activation on A431 cell line, alone or complexed with the Ig-SAP or Mab-ZAP.

B. The anti-HVEM 1.16 mAb does not induce caspase 3/7 activation on A431 cell line.

FIG. 3: General scheme of the biological mechanism according to the present invention.

### EXAMPLE 1

### Material & Methods

HVEM Expression on Prostate Tumor Cells PC3 and DU145 prostate tumor cell lines were obtained from the American Type

Culture Collection Center and were maintained in DMEM (for PC3 cell line) or RPMI (for DU145 cell line) supplemented with 10% fetal bovine serum. These cell lines were phenotyped for HVEM expression using monoclonal anti-HVEM made in-house antibodies. Briefly, cells were incubated for 30 minutes on ice with the appropriate antibody, and then analyzed on LSR-Fortessa cytometer (Becton Dickinson).

HVEM expression was also evaluated on primary prostate tumors. Prostate tumors were obtained at the day of surgery, with consent of patient and agreement of the Institutional Ethic Committee Review Board (Comité d'Orientation Stratégique (COS), Marseille, France). Surgical samples were mechanically dilacerated using scalpels in RPMI. Cell suspensions obtained after nonenzymatic disruption were filtered successively through 70 μm and 30 μm cell strainers (Miltenyi Biotec). Cell suspension was then washed and used directly for flow cytometry staining as described above.

Targeting Through Internalization of HVEM mAbs/Saporin Toxin Complex

Anti-HVEM mAbs purified antibodies were used to study the delivery of toxin (saporin)-conjugated goat anti-mouse IgG secondary antibody (Advanced Targeting Systems) to the A431 human epidermoid carcinoma cell line. This cell line was obtained from the American Type Culture Collection Center and was maintained in DMEM supplemented with 10% fetal bovine serum. A431 cell line was found positive for cell surface HVEM expression by flow cytometry. Briefly, 10000 cells were incubated overnight in flat-bottom 96-well plates. Then, the anti-HVEM primary antibody (with a range from 25 nM to 0.01 nM) and the saporin-conjugated goat anti-mouse IgG secondary antibody (referred as Mab-ZAP, 50 ng) or negative control saporin-conjugated pre-immune goat-IgG antibody (Ig-SAP, 50 ng) were added and the plate was incubated for 48 hours at 37° C. The HVEM mAb/saporin complex is bound by the targeted cells positive for HVEM expression, internalized and saporin is released to inactivate ribosomes. Cell death is evaluated by measuring caspase activity (caspase Glow 3/7 assay luminescence kit (Promega)).

### Results

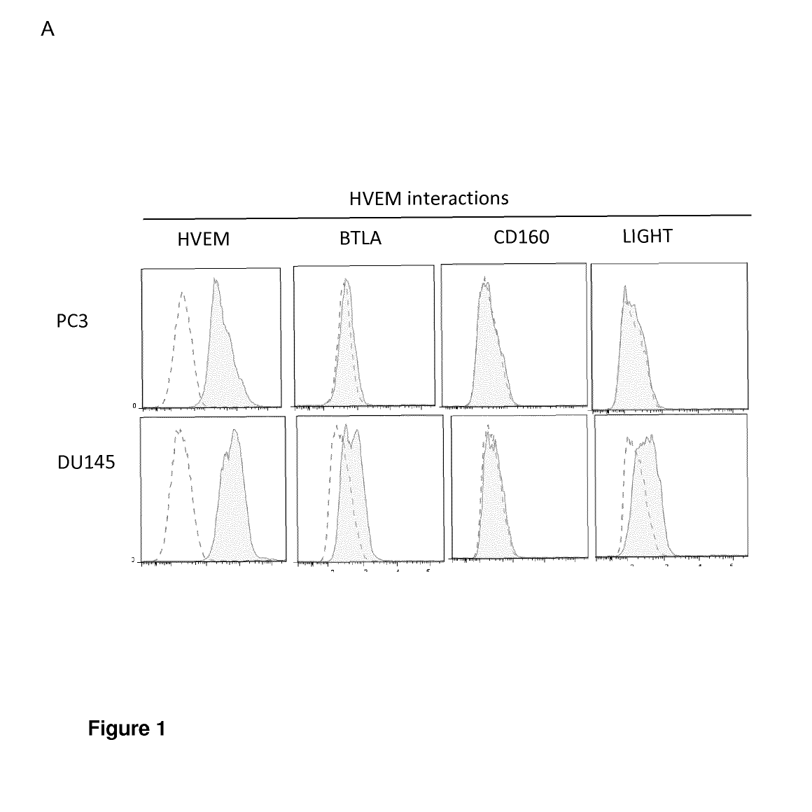

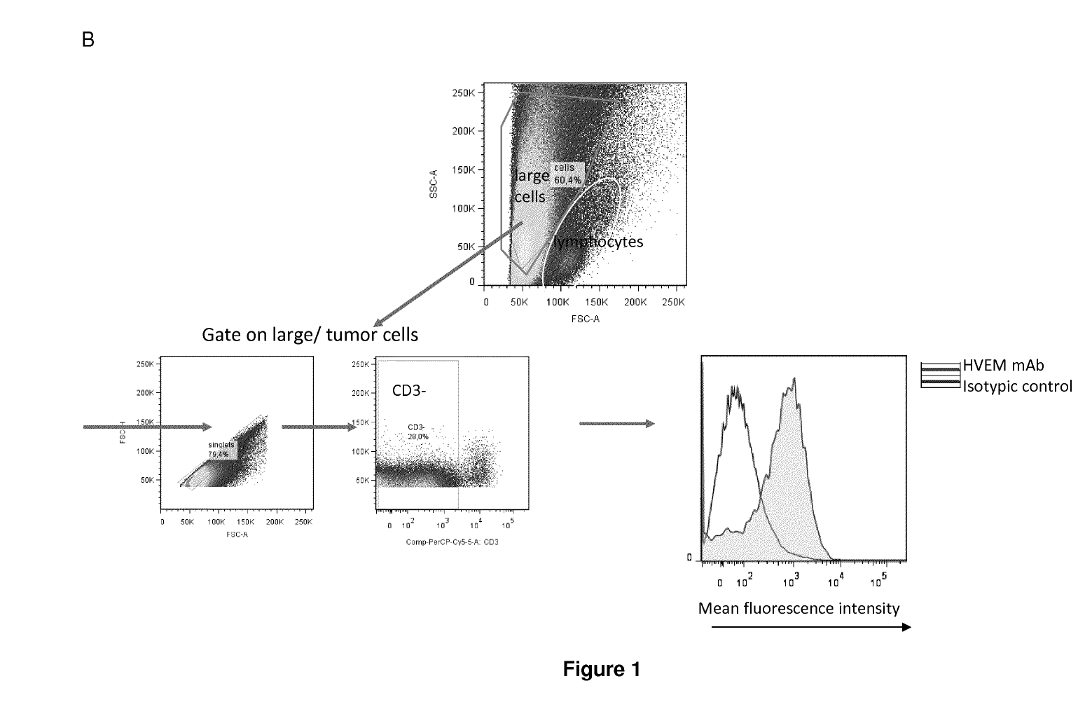

The inventors screened the expression of HVEM and its ligands on prostate tumor cell lines (PC3 and DU145) (FIG. 1A). They found that HVEM was expressed at the cell surface of PC3 and DU145 by flow cytometry, whereas its ligands BTLA, CD160 and LIGHT were not expressed. Then they analysed the expression on tumor cells isolated from human prostate biopsies (FIG. 1B). Briefly, the tumors, collected from the Institut Paoli-Calmettes were dissociated with scalpels, the cell suspension was filtered and the cells were analysed by flow cytometry. Tumor cells were gated on FSC/SSC large cells, negative for the expression of CD3. HVEM was clearly expressed on these prostate tumor cells compared to isotypic control.

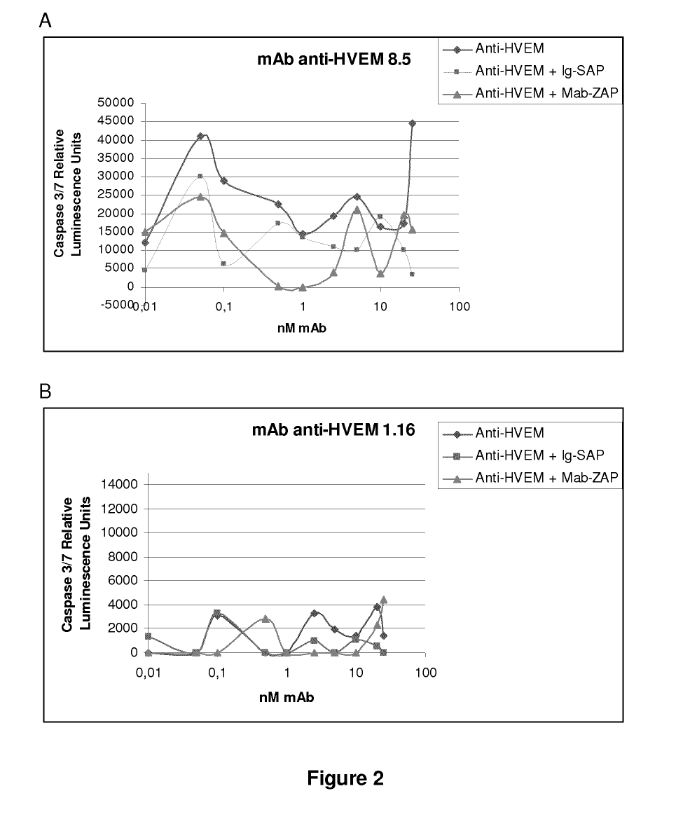

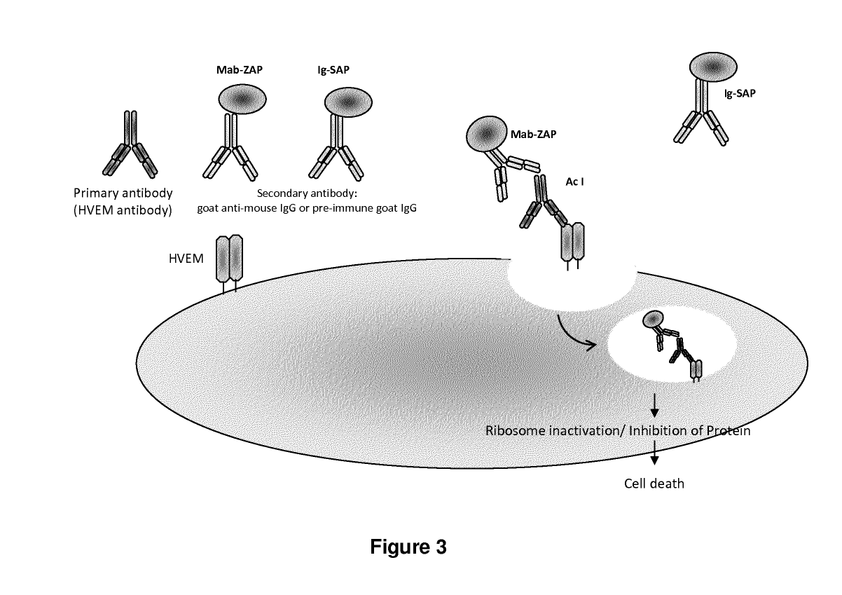

Then, the inventors evaluated which HVEM mAbs were able to induce cell death alone, or in combination with a toxin through internalization of a HVEM mAbs/saporin complex (FIG. 2). The biological mechanism is illustrated in FIG. 3. The HVEM mAb/saporin complex is bound by the targeted cells positive for HVEM expression. The saporin is released into the cells to inactivate ribosomes and induce cell death.

The inventors incubated the A431 cell line with the anti-HVEM primary antibody (with a range from 25 nM to 0.01 nM) and the saporin-conjugated goat anti-mouse IgG secondary antibody (referred as Mab-ZAP, 50 ng) or negative control saporin-conjugated pre-immune goat-IgG antibody (Ig-SAP, 50 ng) for 48 hours at 37° C. Then cell death was evaluated by measuring caspase activity. They found that a group I of HVEM mAbs can induce cell death alone, group II can induce cell death if they are complexed with saporin and internalized, and a third group has no effect on cell death. They observed that the anti-HVEM 8.5 mAb induce caspase 3/7 activation on A431 cell line, alone (caspase activation around 40000 relative luminescence units RLU) or complexed with the Ig-SAP (30000 RLU) or Mab-ZAP (25000 RLU) (FIG. 2A), at a concentration of approximately 0.1 nM. In contrast, the anti-HVEM 1.16 mAb does not induce caspase 3/7 activation on A431 cell line (FIG. 2B).

### EXAMPLE 2

Anti-HVEM purified antibodies are used to study the delivery of toxin (mertansine)-conjugated goat anti-mouse IgG secondary antibody (Advanced Targeting Systems) to the PC3 human prostate tumor cell line or A431 epidermoid carcinoma cell line. Briefly, 5000 cells are incubated overnight in flat-bottom 96-well plates. Then, the anti-HVEM primary antibody (at different concentrations) and the emtansine-conjugated goat anti-mouse IgG secondary antibody or negative control emtansine-conjugated pre-immune goat-IgG antibody are added and the plate is incubated for 48 hours at 37° C. Cell death is evaluated by measuring caspase activity (caspase Glow 3/7 assay luminescence kit (Promega)).

The inventors show that HVEM mAbs can induce cell death if they are complexed with mertansine and internalized.

1-15. (canceled)

16. The hybridoma cell line deposited at the Collection Nationale de Cultures de Microorganismes on May 16, 2013, under the number CNCM I-4751.

17. A monoclonal antibody obtainable from the hybridoma deposited at the Collection Nationale de Cultures de Microorganismes on May 16, 2013, under the number CNCM I-4751.

18. A method for treating a patient suffering from a solid tumor comprising the simultaneous, separate or sequential administration of i) a ligand of HVEM and ii) an immunotoxin.

19. The method according to claim 18, wherein the ligand of HVEM i) is selected from the group consisting of LIGHT, LT, BTLA, CD160, HSV-gD, an anti-HVEM antibody and a fragment or derivative thereof.

20. The method according to claim 18, wherein the ligand of HVEM i) is
an anti-HVEM antibody which binds to HVEM and does not inhibit the binding of BTLA to HVEM, or
an anti-HVEM antibody which inhibits the binding of BTLA to HVEM and which binds to HVEM on an epitope different from the human HVEM sequence CPKCSPGYRVKEACGELTGTVCEPC (SEQ ID NO:1).

21. The method according to claim 18, wherein the ligand of HVEM i) is an anti-HVEM antibody fragment chosen from Fab, Fab′, a F(ab)2, F(ab′)2 and dAb.

22. The method according to claim 18, wherein the ligand of HVEM i) is an anti-HVEM antibody derivative selected from the group consisting of: scFv, (scFv)2, diabodies, multimeric scFv derived from an anti-HVEM antibody and fused to a Fc fragment, whole anti-HVEM antibodies linked together to reach an aggregated form, and antibodies containing at least two Fabs bound face-to-tail.

23. The method according to claim 18, wherein the ligand of HVEM i) is selected from the group consisting of: a monoclonal antibody obtainable from a hybridoma deposited at the Collection Nationale de Cultures de Microorganismes on Apr. 26, 2007, under the number CNCM I-3752; a monoclonal antibody obtainable from the hybridoma deposited at the Collection Nationale de Cultures de Microorganismes on Apr. 26, 2007, under the number CNCM I-3753; a monoclonal antibody obtainable from the hybridoma deposited at the Collection Nationale de Cultures de Microorganismes on Apr. 26, 2007, under the number CNCM I-3754; a monoclonal antibody obtainable from the hybridoma deposited at the Collection Nationale de Cultures de Microorganismes on May 16, 2013, under the number CNCM I-4751; or is a fragment or derivative thereof.

24. The method according to claim 18, wherein the immunotoxin ii) is a chimeric protein made of a modified antibody or antibody fragment, attached to a fragment of a toxin.

25. The method according to claim 18, wherein the immunotoxin ii) comprises a Ribosome Inactivating Protein.

26. The method according to claim 18, wherein the immunotoxin ii) comprises a Ribosome Inactivating Protein selected from the group consisting of saporin, ricin, abrin, gelonin, Pseudomonas exotoxin trichosanthin, luffin, agglutinin and the diphtheria toxin.

27. The method according to claim 18, wherein the toxin is
i) a chemical drug selected from the group consisting of
modeccin, mitogellin, chlortetracycline, mertansine, monomethyl auristatin E, monomethyl auristatin F and enediynes, especially calicheamicins and their related esperamicins;
anticancer agents, preferably chosen from combrestatin, colchicine, actinomycine, duocarmycins and their synthetic analogues, fludarabine, gemcitabine, capecitabine, methotrexate, taxol, taxotere, mercaptopurine, thioguanine, hydroxyurea, cytarabine, cyclophosphamide, ifosfamide, platinum complexes, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, epimbicin, 5-fluorouracil, taxanes, leucovorin, levamisole, irinotecan, estramustine, etoposide, nitrogen mustards, BCNU, nitrosoureas, vinca alkaloids, imatinib mesylate, hexamethylenediamine, topotecan, kinase inhibitors, phosphatase inhibitors, ATPase inhibitors, protease inhibitors, inhibitors of herbimycin A, genistein, erbstatin, and lavendustin A; or
2) a radioisotope selected from the group consisting of 211At, 131I, 125I, 186Re, 188Re, 153Sm, P32, 90Y, 177Lu, 67Cu, 47Sc, 212Bi, 213Bi, 226Th, 111In and 67Ga.

28. The method according to claim 18, wherein said solid tumor is selected from the group consisting of prostate cancer, pancreatic cancer, breast cancer, melanoma, B cell lymphoma, brain cancer, bladder cancer, colon cancer, intestinal cancer, lung cancer, stomach cancer, cervical cancer, ovarian cancer, liver cancer, skin cancer, colorectal cancer, endometrial carcinoma, salivary gland carcinoma, kidney cancer, thyroid cancer, head cancer and neck cancer.


SEQUENCE LISTING (1 sequence)

SEQ ID NO: 1 - protein, 25 aa, Artificial Sequence
  Synthetic epitope
CPKCSPGYRVKEACGELTGTVCEPC